(19) United States
(12) Patent Application Publication
(10) Pub. No.: US 2007/0128342 A1
(43) Pub. Date: Jun. 7, 2007

(54) Method and system for coating a medical device

(76) Inventor: Eric B. Stenzel, Tuam (IE)

Correspondence Address: KENYON & KENYON LLP, 1500 K STREET N.W., SUITE 700, WASHINGTON, DC, 20005, US

(21) Appl. No.: 11/292,567

(22) Filed: Dec. 2, 2005

(51) Int. Cl. A61L 33/00; B05C 5/00

(52) U.S. Cl. 427/2.1; 427/2.24; 118/300

(57) ABSTRACT

A method and device for coating a medical device including the step of heating the medical device and applying frozen ground up particles of coating material to the heated medical device such that the coating material flows on the surface of the medical device and forms a coating thereon.

### FIELD OF THE INVENTION

The present invention relates to a method of coating a medical device.

### BACKGROUND OF THE INVENTION

Medical implants are used for a number of medical purposes, including the reinforcement of recently re-enlarged lumens, the replacement of ruptured vessels, and the treatment of disease such as vascular disease by local pharmacotherapy, i.e., delivering therapeutic drug doses to target tissues while minimizing systemic side effects. Such localized delivery of therapeutic agents has been achieved using medical implants, which both support a lumen within a patient's body and place appropriate coatings containing absorbable therapeutic agents at the implant location. Examples of such medical devices include catheters, guide wires, balloons, filters (e.g., vena cava filters), stents, stent grafts, vascular grafts, intraluminal paving systems, implants and other devices used in connection with drug-loaded polymer coatings. Such medical devices are implanted or otherwise utilized in body lumina and organs such as the coronary vasculature, esophagus, trachea, colon, biliary tract, urinary tract, prostate, brain, and the like.

The process of applying a coating onto a medical device, such as a stent, may be accomplished by a number of methods including, for example, spray coating, spin-coating, and electrostatic deposition. The spray-coating method has been frequently used because of its excellent features, e.g., good efficiency and control over the amount or thickness of coating. However, the conventional spray-coating methods, which are usually implemented with a device such as an airbrush or nozzle, have drawbacks. For example, conventional spraying methods are inefficient. In particular, generally only 5% of the coating solution that is sprayed to coat the stent is actually deposited on the surface of the stent. The majority of the sprayed coating solution is therefore wasted.

Therefore, there is a need for an improved method for coating medical devices that reduces waste material volume and coating cost.

### SUMMARY OF THE INVENTION

The present invention concerns methods and apparatus for providing a coating on a structure. In an exemplary embodiment, the present invention is directed to a medical device adapted for insertion into a body lumen wherein the medical device is coated with an active substance and a bio-compatible polymer for binding the active substance to the structure. In another exemplary embodiment, the present invention provides a method of manufacturing a medical device having a coating.

In an exemplary embodiment of the invention, a medical device, such as a stent, may be coated with a coating material by (i) controlling the temperature of at least one of the medical device and the coating material such that upon contact with the medical device the coating material coats the medical device; and (ii) applying the coating material to the medical device in a solid state.

In an exemplary embodiment of the invention, the medical device may be heated and the coating material may be cooled such that the coating material is applied to the heated medical device in a frozen state and melts on the medical device.

In an exemplary embodiment of the invention, the coating material may include a mixture of at least one solvent and at least one polymer.

In an exemplary embodiment of the invention, the coating material may include at least one therapeutic agent.

In an exemplary embodiment of the invention, the component compounds of the coating solution may be individually frozen, ground to fine powders, and mixed to provide a powdered homogeneous mixture.

In an exemplary embodiment of the invention, as an initial step, the medical device may be suspended in a coating chamber.

In an exemplary embodiment of the invention, coating material that misses the medical device and/or falls off the medical device may be captured and reused.

In an exemplary embodiment of the invention, the temperatures of the medical device and the coating material may be controlled such that solvents in the coating material vaporize only after the coating material has had a chance to melt and flow sufficiently to provide a smooth coating on the medical device.

In an exemplary embodiment of the invention, the temperatures of the medical device and the coating material may be controlled such that solvents vaporize approximately between 2 and 240 minutes, for example, between 2 and 4 minutes, after application of the coating material to the medical device.

In an exemplary embodiment of the invention, the temperatures of the medical device and the coating material may be controlled so as to prevent droplets of coating material from forming on the medical device.

In an exemplary embodiment of the invention, the temperatures of the medical device and the coating material may be controlled such that the coating material takes between approximately 1 millisecond and 10 minutes, for example, between 1 and 1000 milliseconds, to melt.

In an exemplary embodiment of the invention, the medical device may be rotated during application of the coating material.

In an exemplary embodiment of the invention, the coating material may include ground up coating particles applied to the medical device (i) via a gas assisted spray process, (ii) via electrostatic deposition, or (iii) by dropping the particles onto the medical device.

In an exemplary embodiment of the invention, the gas assisted spray process may use helium or nitrogen or another suitable gas to carry the ground up coating particles to the medical device.

In an exemplary embodiment of the invention, the temperature of the medical device may be monitored and heat may be added to the medical device when its temperature falls below a predetermined temperature.

In an exemplary embodiment of the invention, the temperature of the medical device may be monitored via at least one of thermal imaging, a thermocouple and detecting a change in the resistivity of the medical device.

In an exemplary embodiment of the invention, the medical device may be heated by at least one of (i) passing a current therethrough, (ii) exposing the medical device to radio frequency, (iii) exposing the medical device to a heated stream of gas, (iv) exposing the medical device to laser light, (v) exposing the medical device to infra-red radiation, (vi) exposing the medical device to a heating element, and (vii) exposing the medical device to particle or microwave radiation.

In an exemplary embodiment of the invention, the coating material may be cooled by at least one of (i) cooling a chamber containing the medical device and (ii) cooling a container holding the coating material.

In an exemplary embodiment of the invention, another coating material may be applied after application of the coating material. The coating material first applied to the medical device may include at least one polymer without therapeutics and the other coating material may include at least one therapeutic.

In an exemplary embodiment of the invention, the medical device may be dried after application of the coating.

In an exemplary embodiment of the invention, the therapeutic agent is Paclitaxel.

An exemplary system of the present invention for coating a medical device is configured to perform the method of the present invention as described above. The system may include a source of coating material in a solid state, a delivery device, configured to direct a stream of the coating material onto the medical device, and a heating source for heating the medical device to a temperature sufficient to assure that the coating material is rendered flowable via contact with the medical device. The system may also include a cooler for cooling the coating material.

### BRIEF DESCRIPTION OF THE DRAWING

The present invention will become more fully understood from the detailed description given hereinbelow and the accompanying drawing, which is given by way of illustration only and wherein:

FIG. 1 is a schematic illustration of a system according to the present invention for coating a medical device.

### DETAILED DESCRIPTION OF THE INVENTION

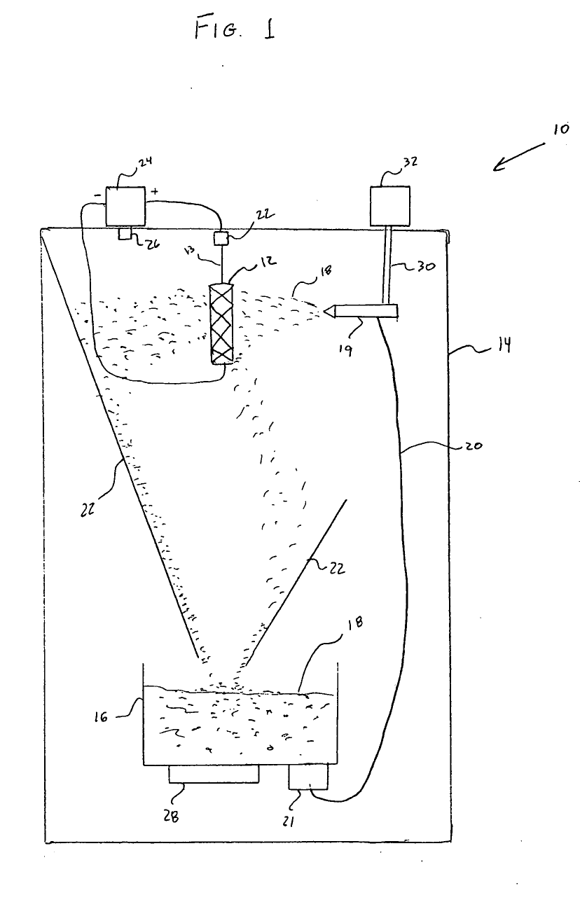

FIG. 1 illustrates an exemplary embodiment of a system 10 according to the present invention for coating a device, such as a medical device. Non-limiting examples of medical devices according to the present invention include catheters, guide wires, balloons, filters (e.g., vena cava filters), stents, stent grafts, vascular grafts, intraluminal paving systems, implants and other devices used in connection with coatings, e.g., drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

FIG. 1 illustrates a stent 12 suspended vertically in a chamber 14 over a container 16 used to store coating material 18 for coating the stent 12. A spray or nozzle 19, communicating with the container 16 via conduit 20 and with a gas supply 32 via conduit 30, is used to spray the coating material 18 onto the stent. Pump 21 may be used to drive the coating material 18 towards the spray or nozzle 19. Coating material 18 which misses the stent 12 or falls off the stent 12 is directed back into container 16 via a guide or chute 22 located below and to the sides of the suspended stent 12. Although shown suspended vertically, stent 12 may also be suspended horizontally or in any other suitable orientation. Further, stent 12 may be suspended from a wire 13 allowing for manual rotation of the stent or connected to motor 22 providing for motorized rotation of the stent 12. Wire 13 and/or motor 22 may also be used to move the stent 12 in the vertical direction. Further, gas supply 32 may be configured with a motor to move spray or nozzle 19.

Gas supply 32 supplies a carrier gas such as, for example, oxygen or nitrogen for carrying the particles of coating material 18 across the space between the stent 12 and the spray or nozzle 19. The pressure of the gas supply 32 may be controlled so as to prevent damage to the stent 12 and to maximize the amount of coating material 18 that sticks to the stent 12, i.e., the pressure may be controlled to minimize the amount of coating material 18 that either flies right past the stent 12 or falls off the stent 12. In an exemplary embodiment of the present invention, the gas supply 32 supplies gas at an operating pressure of between 0.2 bars and 1 bar.

Stent 12 is maintained at a temperature sufficient to render the coating material sprayed onto the stent 12 flowable so as to coat the stent 12. The coating solution and stent 12 may be heated, for example, to a temperature of −95 degrees Centigrade, for example, using a power supply 24, with the room temperature at, for example, 22 degrees Centigrade. Alternatively, stent 12 may be heated using other known heating methods, for example, using RF energy, laser, infra-red heating, heating element with or without a fan, etc. Stent 12 may be heated to a preset temperature before application of the coating material 18 and allowed to fluctuate in temperature during application of the coating material 18. Alternatively, the temperature of stent 12 may be controlled using, for example, a feedback loop, so as to assure that it remains steady or within a predetermined range during application of the coating material 18. A means for monitoring the temperature 26, shown schematically in FIG. 1, including for example, a thermal imaging device, a thermocouple or a controller used to sense a change in resistivity, may be used to monitor the temperature of stent 12.

Coating material 18 may include a mixture of at least one solvent and at least one polymer. Coating material 18 may also include at least one therapeutic agent. The mixture making up the coating material 18 may be solidified and formed into solid particles, for example, by grinding. The solid particles become flowable when sprayed onto stent 12. For example, the mixture making up coating material 18 may be frozen and ground up so as to produce particles, e.g., having a diameter of 1 μm to 10 μm, which melt upon contact with the stent 12. As indicated above, the use of a solid coating material increases the efficiency of the coating process as unused coating material 18 falls into container 16 and is reusable rather than sticking to side walls of the chamber 14 and escaping from openings in the chamber 14. Cooler 28 may be used to cool and maintain the coating material 18 in a frozen state. Alternatively, or in combination with cooler 28, another cooler may be used to cool the entire chamber 14. After application of the coating material 18, the stent 12 may be dried.

The temperatures of the stent 12 and the coating material 18 may be controlled so as to prevent droplets of coating material 18 from forming on the stent 12 while at the same time assuring that solvents in the coating material 18 vaporize only after the coating material 18 has had a chance to melt and flow sufficiently to provide a smooth coating on the stent 12. Maintaining the stent 12 and/or the coating material 18 too cold may lead to the gathering of too much coating material 18 on the stent 12, which may form undesirable droplets and lead to dripping, whereas maintaining them too hot may lead to premature evaporation of the coating material solvents.

The coating may typically range from about 1 to about 50 microns thick. In the case of balloon catheters, the thickness may be from about 1 to about 10 microns, for example, from about 2 to about 5 microns. Very thin polymer coatings, such as about 0.2-0.3 microns, and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

In an exemplary embodiment of the present invention, the temperatures of the stent 12 and the coating material 18 are controlled such that the coating material 18, after application to the stent 12, takes between approximately 1 millisecond and 10 minutes, for example, between 1 and 1000 milliseconds, to melt, and such that it takes approximately 2 to 240 minutes, for example, 2 to 4 minutes, for a predetermined portion of the solvent in the coating material 18 to vaporize. If a drug is included in the coating material 18 the temperature may be controlled so as to prevent damage to the drug. For example, if Paclitaxel is used the temperature may be kept below 80° C.

The drug optionally included in the coating material 18 may be any pharmaceutically acceptable therapeutic agents such as non-genetic therapeutic agents, biomolecules, small molecules, or cells. Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and CDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

The polymers included in the coating material 18 may be biodegradable or non-biodegradable. Non-limiting examples of suitable non-biodegradable polymers include polystrene; polyisobutylene copolymers and styrene-isobutylene block copolymers such as styrene-isobutylene-styrene tri-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Any of the above mentioned therapeutic agents may be incorporated into a polymeric coating on the stent 12 or applied onto a polymeric coating on the stent 12. More specifically, the therapeutic agent may be added to the coating material mixture and then frozen and ground up, as detailed above, or may be applied as a separate layer over the applied coating mixture. The therapeutic agent may be independently frozen and ground up and separately applied, for example, via spraying, to the stent 12.

The coating material 18 used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

Solvents may also be utilized in any order. For example, an initial polymer/solvent mixture can be formed and then the drug added to the polymer/solvent mixture. Alternatively, the polymer, solvent, and drug can be added simultaneously to form a mixture. Furthermore, multiple types of drug, polymers, and/or solvents may be utilized.

The stent 12 may also contain a radio-opacifying agent within its structure to facilitate viewing the stent 12 during insertion and at any point while the device is implanted. Non-limiting examples of radio-opacifying agents are bismuth subcarbonate, bismuth oxychloride, bismuth trioxide, barium sulfate, tungsten, and mixtures thereof.

The coating material 18 may be applied to stent 12 using other delivery techniques. For example, coating material 18 may be delivered using a gravity flow process in which container 16 is placed over stent 12 and a controlled amount of coating material is released onto the stent 12, for example, by tipping the container 16 or opening a container door. Alternatively, a conveyor belt may be used to deliver coating material 18 from container 16 to a release point over stent 12. In yet another embodiment, coating material 18 may be delivered using electrostatic depostion, as described, for example, in U.S. Pat. Nos. 5,824,049 and 6,096,070 to Ragheb et al., herein incorporated by reference in their entirety. A surface of the stent 12 may be grounded and the particles of the coating material 18 may be charged. Since the particles are charged, when they are applied to the surface of the stent 12, they will be attracted to the surface since it is grounded.

The foregoing description and example have been set forth merely to illustrate the invention and are not intended as being limiting. Each of the disclosed aspects and embodiments of the present invention may be considered individually or in combination with other aspects, embodiments, and variations of the invention. None of the steps of the methods of the present invention are confined to any particular order of performance. Modifications of the disclosed embodiments incorporating the spirit and substance of the invention are within the scope of the present invention.

We claim:

1. A method for coating a medical device with a coating material comprising the steps of:
a) controlling a temperature of at least one of the medical device and the coating material such that contact with the medical device transforms the coating material from a solid state to a fluid state; and
b) applying the coating material to the medical device in the solid state.

2. The method of claim 1, wherein the medical device is heated and the coating material is cooled, the coating material is applied to the heated medical device in a frozen state and melts on the medical device.

3. The method of claim 1, wherein the medical device is a stent.

4. The method of claim 1, wherein the coating material comprises one of (i) a mixture of at least one solvent and at least one polymer and (ii) a mixture of at least one solvent, at least one polymer and at least one therapeutic agent.

5. The method of claim 1, further comprising the step of capturing coating material that does not adhere to the medical device.

6. The method of claim 1, wherein the temperatures of the medical device and the coating material are controlled such that solvents in the coating material vaporize only after the coating material has had a chance to melt and flow sufficiently to provide a smooth coating on the medical device.

7. The method of claim 1, wherein the coating material comprises ground up coating particles applied to the medical device via one of (i) a gas assisted spray process, (ii) electrostatic deposition, and (iii) by dropping the particles onto the medical device.

8. The method of claim 1, wherein the temperature of the medical device is monitored and a heating rate of the medical device is controlled so as to maintain the medical device within a predetermined temperature range.

9. The method of claim 1, further comprising the step of applying another coating material after application of the coating material, the coating material first applied to the medical device comprising at least one polymer without therapeutic and the other coating material comprising at least one therapeutic.

10. A system for coating a medical device, comprising:
a source of coating material in a solid state;
a delivery device configured to direct a stream of the coating material onto the medical device; and
a heating source for heating the medical device to a temperature sufficient to assure that the coating material is rendered flowable via contact with the medical device.

11. The system of claim 10, further comprising a control device configured to control the temperature of the medical device such that upon contact with the medical device the coating material coats the medical device.

12. The system of claim 10, wherein the medical device is a stent.

13. The system of claim 10, wherein the coating material comprises one of (i) a mixture of at least one solvent and at least one polymer and (ii) a mixture of at least one solvent, at least one polymer and at least one therapeutic agent.

14. The system of claim 10, further comprising a bin and a guide element configured to guide coating material that does not adhere to the medical device into the bin.

15. The system of claim 11, wherein the control device is configured to control the temperatures of the medical device and the coating material such that solvents in the coating material vaporize only after the coating material has had a chance to melt and flow sufficient to provide a smooth coating on the medical device.

16. The system of claim 10, where the coating material comprises ground up frozen solid coating particles.

17. The system of claim 16, wherein the delivery device comprises one of (i) a gas assisted spray device, (ii) an electrostatic deposition device, and (iii) a hopper above the medical device.

18. The system of claim 11, wherein the control device further comprises a temperature monitor, the heating source being configured to add heat to the medical device when its temperature as monitored by the temperature monitor falls below a predetermined temperature.

19. The system of claim 10, further comprising a cooler is configured to cool the coating material by cooling at least one of a chamber containing the medical device and cooling a container holding the coating material.

20. The system of claim 10, further comprising a source of a second coating material, the coating material comprising at least one polymer without therapeutic and the second coating material comprising at least one therapeutic.

21. A device for coating a stent, comprising:
a source of ground up frozen coating material; and
a delivery means for directing a stream of the frozen coating material onto the stent.

22. The device of claim 21, further comprising:
a cooler-means for maintaining the frozen coating material in a frozen state prior to application of the coating material onto the stent.

23. The device of claim 21, further comprising:
a heater means for heating the stent and maintaining the stent within a predetermined temperature range at least during application of the coating material, said heater means heating the stent sufficiently to assure melting of the coating material in contact with the stent and not obstructing the stream of the coating material.

24. A medical device for insertion into a body prepared according to the method of claim 1.
